SEQUENCE LISTING (3 sequences)

SEQ ID NO: 1 - DNA, 19 bp, synthetic construct
  source 1..19: mol_type = other DNA; note = synthetic DNA used as primer
tccgtaggtgaacctgcgg

SEQ ID NO: 2 - DNA, 20 bp, synthetic construct
  source 1..20: mol_type = other DNA; note = synthetic DNA used as primer
tcctccgcttattgatatgc

SEQ ID NO: 3 - DNA, 562 bp, Kluyveromyces marxianus
  source 1..562: mol_type = genomic DNA
ccaacggggattgccttagtacggcgagtgaagcggcaaaagctcaaatttgaaatctggcgtcttcgacgtccgagttgtaatttgaagaaggcgactttgtagctggtccttgtctatgttccttggaacaggacgtcatagagggtgagaatcccgtgtggcgaggatcccagttatttgtaaagtgctttcgacgagtcgagttgtttgggaatgcagctctaagtgggtggtaaattccatctaaagctaaatattggcgagagaccgatagcgaacaagtacagtgatggaaagatgaaaagaactttgaaaagagagtgaaaaagtacgtgaaattgttgaaagggaagggcatttgatcagacatggcgtttgcttcggctttcgctgggccagcatcagttttagcggttggataaatcctcgggaatgtggctctgcttcggtagagtgttatagcccgtgggaatacagccagctgggactgaggattgcgacttttgtcaaggatgctggcgtaatggttaaatgccgcccgtcttgaacccacggacca

(19) United States
(12) Patent Application Publication
(10) Pub. No.: US 2026/0308509 A1
(43) Pub. Date: Oct. 8, 2026

(54) KLUYVEROMYCES MARXIANUS AND USE THEREOF

(71) Applicant: ANGEL YEAST CO., LTD., Yichang, Hubei (CN)

(72) Inventors: Yafang SUN, Yichang, Hubei (CN); Xianwu QIN, Yichang, Hubei (CN); Tianfen GUO, Yichang, Hubei (CN); Yan ZHANG, Yichang, Hubei (CN); Yu SHI, Yichang, Hubei (CN)

(21) Appl. No.: 19/483,190

(22) PCT Filed: Oct. 25, 2023
(86) PCT No.: PCT/CN2023/126403

§ 371 Date: Nov. 10, 2025

(30) Foreign Application Priority Data
May 13, 2023 (CN) 202310540115.9

(51) Int. Cl. C12N 1/16; A23J 3/20; C12R 1/85

(52) U.S. Cl. CPC C12N 1/16; A23J 3/20; C12R 2001/85

(57) ABSTRACT

The present invention relates to the technical field of fermentation, and provides Kluyveromyces marxianus and a use thereof. The accession number of Kluyveromyces marxianus is CCTCC No: M2023217. The original strain has good lactic acid resistance and low pH value tolerance. The strain obtained by mutagenesis by using the original strain has organic acid resistance and low pH value tolerance. For example, the original strain can tolerate lactic acid having a concentration of 5.5 wt % and can grow under the condition that the pH value is 2, and particularly, the tolerance of the original strain to lactic acid having a concentration of 4.5 wt % to 5.5 wt % reaches 80%-90%. The original strain can effectively transform lactic acid into microbial proteins, has more efficient lactic acid utilization capacity in corn steep liquor fermentation, can increase the additional value of the corn industry, achieves high-value utilization of low biomass resources, reduces, as an alternative protein, the pressure on environmental resources caused by the traditional breeding industry, meets the protein requirements of global population and the field of feed, and achieves economic development and environmental protection of the fermentation industry.

### CROSS-REFERENCE TO RELATED APPLICATION

The present application is a U.S. National Phase of International Application No. PCT/CN2023/126403, filed with the State Intellectual Property Office of P. R. China on Oct. 25, 2023, which claims the priority of Chinese Patent Application No. 202310540115.9 filed with the Chinese National Intellectual Property Office on May 13, 2023. The entire contents of all above-mentioned applications are incorporated herein by reference.

### SEQUENCE LISTING

The application contains a Sequence Listing which is hereby incorporated by reference in its entirety. Said .xml copy, created on, Oct. 17, 2025, is named PICN-US250028AQ-20251022-Sequence_Listing.xml, and is 5 kb in size.

### TECHNICAL FIELD

The present invention relates to the technical field of fermentation, and more particularly, to Kluyveromyces marxianus and a use thereof.

### BACKGROUND ART

By 2050, the world's population is expected to reach 9 billion, and the contradiction between food access and population growth will remain prominent, forming a huge “protein gap” between globally available proteins and expected demands in the next 30 years, with an estimated protein gap of 250 million tons by 2050. Proteins are mainly derived from animal proteins and plant proteins, but both animal proteins and plant proteins need to be bred or planted in certain land areas. However, land resources are facing problems such as land destruction, water and soil erosion, soil salinization and desertification. Therefore, alternative protein sources need to be developed to meet the protein requirements of nearly 9 billion people.

At present, alternative proteins have become an important topic in the food industry. The global demand for alternative proteins is surging, and the total scale of the alternative protein market will grow at a double-digit rate every year, and is expected in the market to reach 2.6 billion yuan in 2023. Common alternative proteins mainly include microorganisms and algae. Compared with animal and plant proteins, microbial proteins have the advantages of nutritional comprehensiveness, clean labels, sustainable environmental friendliness, etc. The microorganisms generally used for the production of microbial proteins include bacteria, fungi and protists, among which a yeast protein is a typical representative, with non-negligible potential ability and development space, and the most popular yeast species are Saccharomyces cerevisiae, Candida oleophila, Kluyveromyces marxianus, etc. Kluyveromyces marxianus is an unconventional yeast commonly found in dairy products. Kluyveromyces marxianus was approved as a new food ingredient in China as early as 2013 and was approved to be added to the “List of Cultures Available in Food” in 2022. This strain has the advantages of broad-spectrum substrate utilization, heat resistance, fast growth rate, special flavor, etc., and can be used in biofuels, brewing, dairy products, condiments, baking, feed and other fields.

In recent years, with the rapid development of animal husbandry in China, feed resources are becoming increasingly scarce, and abundant low-value biomass such as grain processing by-products, waste residues and agricultural and sideline product wastes are discarded and incinerated, resulting in waste of resources. Therefore, achieving high-value utilization of biomass is an inevitable trend in the development of feed. China is a major producer of corn starch. Corn steep liquor, as one of the by-products in the wet production process of corn starch, is rich in sugars, soluble proteins, a variety of amino acids, polypeptides, vitamins and other organic compounds, but is limited in application due to the presence of components such as lactic acid. Therefore, the development of Kluyveromyces marxianus that can tolerate and be efficiently transformed into microbial proteins using lactic acid can improve the additional value of the corn industry, achieve the high-value utilization of low biomass resources, reduce, as an alternative protein, the pressure on environmental resources brought by the traditional aquaculture industry, alleviate the protein requirements of the global population and the field of feed, has better adaptability in food and industrial high-temperature fermentation environments compared with Saccharomyces cerevisiae, can reduce the cooling cost, and achieve the economic development and environmental protection of the fermentation industry. It can be seen that Kluyveromyces marxianus is a microorganism with a great developing potential.

Chinese patent application CN115287202A discloses a Kluyveromyces marxianus strain and a use thereof in the preparation of functional feed, wherein Kluyveromyces marxianus is made into a fungal solution form. The fermentation microbial agent also includes bacterial solutions of Bacillus subtilis and Bacillus coagulans and is used for performing solid-state fermentation on a solid fermentation medium containing traditional Chinese medicine residues. The functional feed can effectively promote animal growth, improve animal immunity, reduce diarrhea rate and mortality rate, improve the feed conversion rate, also regulate the metabolism, promote growth, and improve meat quality when used as livestock and poultry feed or a feed additive. It is a functional protein feed which is prepared by taking radix bupleuri residues as a main raw material and performing solid-state fermentation in the form of a compound microbial agent. The viable count, pH, odor, moisture, polysaccharides, crude proteins, crude fibers, organic acids and other indexes of each bacterium in the fermentation process are detected, and a feeding test is carried out to investigate the effect on the growth performance of fattening pigs. However, the characteristics of Kluyveromyces marxianus are not described in detail, so it is not clear whether Kluyveromyces marxianus can improve a fermentation effect and can only be applied in the field of functional feed.

Chinese patent application CN115211490A provides a new industrial mass production mode of a yeast culture in which the yeast culture is prepared by taking Kluyveromyces marxianus as a main microorganism by means of a microbial and enzyme synergistic fermentation technology. The yeast culture contains aromatic substances such as alcohols and esters and flavoring substances such as nucleotides, polypeptides and free amino acids, has unique flavor and nutritional characteristics, can increase the food consumption of animals and is beneficial to the improvement of production performances, and compared with a Saccharomyces cerevisiae culture, solves the problems that after fermentation, wet cooking wine is strong in taste, and animal ingestion is affected. The yeast culture is widely used in animal feed, and can significantly enhance the antioxidant function and immunity of animal bodies, promote the animal growth and improve the animal product quality. However, the crude protein content in this microbial agent is 19.84% after the addition of complex enzymes, and the substrate utilization and protein production ability of a Kluyveromyces marxianus strain are not evaluated.

Chinese patent application CN 107287127A discloses a strain of lactic acid-tolerant ester-producing yeast, which is classified and named Pichia kudriavzevii, is capable of tolerating 0-15% lactic acid and metabolizing to generate a plurality of ethyl ester volatile flavor substances, such as ethyl acetate, ethyl propionate and phenethyl acetate in an environment of 0-12% lactic acid, is a brewing functional strain with excellent performances and is only applied in the field of brewing.

Chinese patent CN 113583883B discloses a strain of ester-producing Debaryomyces hanseni which is isolated from Castanea mollissima. This strain has the properties of high temperature resistance, lactic acid resistance, ethanol resistance, high ester production, etc. Specific indexes are as follows: this strain is capable of tolerating a high temperature of 50° C., has lactic acid tolerance of 2%, ethanol tolerance of 8%, and ester production of 1.7 g/L, can be applied to the fermentation of brewed wine, and can effectively improve the flavor and quality of finished brewed wine, but can only be used in the field of winemaking.

### SUMMARY OF THE INVENTION

In view of the above technical problems existing in the prior art, the present application provides Kluyveromyces marxianus and a use thereof. This strain can tolerate and be efficiently transformed into microbial proteins using lactic acid, can improve the additional value of the corn industry, achieve the high-value utilization of low biomass resources, reduce, as an alternative protein, the pressure on environmental resources brought by the traditional aquaculture industry, alleviate the protein requirements of global population and field of feed, and achieve the economic development and environmental protection of the fermentation industry.

Specifically, the present application provides the following technical solutions.

The present application provides Kluyveromyces marxianus, the accession number of the Kluyveromyces marxianus being CCTCC No: M 2023217.

The present application provides mutant Kluyveromyces marxianus, wherein the biomass of the mutant Kluyveromyces marxianus is increased by more than 5%, preferably more than 10%, and further preferably 10-30%, compared with the above-mentioned Kluyveromyces marxianus.

Preferably, for the mutant Kluyveromyces marxianus, the protein content of the mutant Kluyveromyces marxianus is increased by more than 5%, preferably more than 10%, and further preferably 15-25%, compared with the above-mentioned Kluyveromyces marxianus with the accession number of CCTCC No: M 2023217.

Preferably, for the Kluyveromyces marxianus, the accession number of the Kluyveromyces marxianus is CCTCC No: M 20222110.

Preferably, for the Kluyveromyces marxianus, the Kluyveromyces marxianus has tolerance to an organic acid, and preferably, the organic acid is lactic acid.

Preferably, for the Kluyveromyces marxianus, a tolerance concentration of the Kluyveromyces marxianus to the lactic acid is less than 5.5 wt %, preferably 1-5.5 wt %.

Preferably, for the Kluyveromyces marxianus, the Kluyveromyces marxianus has tolerance to a low pH value.

Preferably, for the Kluyveromyces marxianus, the Kluyveromyces marxianus uses an organic acid as a carbon source.

The present application provides a use of the above-mentioned Kluyveromyces marxianus in the production of microbial proteins.

The present application provides a use of the above-mentioned Kluyveromyces marxianus in high-value transformation of an agricultural and forestry product, and preferably, the agricultural and forestry product is corn steep liquor.

The present application provides a microbial agent. The microbial agent includes the above-mentioned Kluyveromyces marxianus.

The present application provides a fermentation composition, which is obtained by fermenting the above-mentioned Kluyveromyces marxianus.

The application provides a method for producing a microbial protein. The method includes:
fermenting the above-mentioned Kluyveromyces marxianus or the above-mentioned microbial agent to obtain the microbial protein.

The present application provides a food. The food contains the above-mentioned fermentation composition or the microbial protein obtained by the above-mentioned method.

The present application provides feed. The feed contains the above-mentioned fermentation composition or the microbial protein obtained by the above-mentioned method.

The present application provides a use of the above-mentioned original strain or mutant strain in feed production.

The present application has the following beneficial effects.

The AMCC 30634 strain and the AMCC 31342 strain of the present application have low pH tolerance, lactic acid tolerance and utilization capacity, and can effectively transform lactic acid into microbial proteins. In particular, the mutant AMCC 31342 strain can tolerate lactic acid having a pH of 2.0 and a concentration of 5%, and has tolerance up to 80-90% under the condition that the concentration of the lactic acid is 4.5-5.5%, further indicating that this strain has more efficient lactic acid utilization capacity in corn steep liquor fermentation, can increase the additional value of the corn industry, achieves high-value utilization of low biomass resources, reduces, as an alternative protein, the pressure on environmental resources caused by the traditional breeding industry, relieve the protein requirements of global population and the field of feed, and achieves economic development and environmental protection of the fermentation industry.

### Strain Preservation Information

The Kluyveromyces marxianus strain used in the present application is preserved in the China Center for Type Culture Collection (CCTCC) on Feb. 27, 2023, the accession number is CCTCC No: M 2023217, the accession address is Wuhan University, Wuhan, China, the postal code is 430072, and the telephone is 027-68754052.

The Kluyveromyces marxianus obtained by mutagenesis in the present application is preserved in the China Center for Type Culture Collection (CCTCC) on Dec. 30, 2022, the accession number is CCTCC No: M 20222110, the accession address is Wuhan University, Wuhan, China, the postal code is 430072, and the telephone is 027-68754052.

### BRIEF DESCRIPTION OF THE DRAWINGS

FIG. 1 shows a schematic diagram of a colony morphology of Kluyveromyces marxianus.

FIG. 2 shows a schematic diagram of a morphology of Kluyveromyces marxianus under a microscope.

FIG. 3 is a schematic diagram of growth curves of a mutant strain and an original strain at different pH values.

FIGS. 4A to 4C are schematic diagrams of growth curves of the mutant strain and the original strain in YPD mediums containing different concentrations of lactic acids, wherein FIG. 4A is a schematic diagram of growth curves of the mutant strain and the original strain in a YPD medium containing 2% lactic acid, FIG. 4B is a schematic diagram of growth curves of the mutant strain and the original strain in a YPD medium containing 4% lactic acid, and FIG. 4C is a schematic diagram of growth curves of the mutant strain and the original strain in a YPD medium containing 5% lactic acid.

FIG. 5 is a schematic diagram of growth curves of the mutant strain and the original strain in a carbon source medium containing 2% lactic acid.

FIG. 6 is a schematic diagram of growth curves of the mutant strain and the original strain in a corn steep liquor-simulated fermentation medium.

### DETAILED DESCRIPTION OF THE INVENTION

The present application provides Kluyveromyces marxianus, wherein the accession number of the Kluyveromyces marxianus is CCTCC No: M 2023217.

The above-mentioned strain of the present application is a strain which is isolated from samples of fermented dairy products in the Tibet Autonomous Region. This strain has excellent resistance to an organic acid and can grow using lactic acid as a carbon source. In addition, this strain has low pH tolerance.

In the present application, the above-mentioned strain with the accession number of CCTCC No: M 2023217 is subjected to mutagenesis as an original strain to obtain a strain with better performances. In the present application, a method for mutagenesis is not limited in the present application in any way. A person skilled in the art can choose conventional methods for mutagenesis. For example, an ARTP mutagenesis method may be used for mutagenesis. That is, strains may be subjected to mutagenesis at different times to screen the strains.

The original strain refers to an originating strain used for breeding treatment. In the present application, the above-mentioned strain with the accession number of CCTCC No: M 2023217 is subjected to mutagenesis as an original strain to obtain a mutant strain with better performances.

In the present application, an ITS sequence of the above-mentioned strain with the accession number of CCTCC No: M 2023217 is shown in SEQ ID NO: 3 as follows: CCAACGGGGATTGCCTTAGTACGGCGAGTGAAGCGGCAAAAGCTCAAATT TGAAATCTGGCGTCTTCGACGTCCGAGTTGTAATTTGAAGAAGGCGACTTT GTAGCTGGTCCTTGTCTATGTTCCTTGGAACAGGACGTCATAGAGGGTGAG AATCCCGTGTGGCGAGGATCCCAGTTATTTGTAAAGTGCTTTCGACGAGTC GAGTTGTTTGGGAATGCAGCTCTAAGTGGGTGGTAAATTCCATCTAAAGCT AAATATTGGCGAGAGACCGATAGCGAACAAGTACAGTGATGGAAAGATGA AAAGAACTTTGAAAAGAGAGTGAAAAAGTACGTGAAATTGTTGAAAGGG AAGGGCATTTGATCAGACATGGCGTTTGCTTCGGCTTTCGCTGGGCCAGCA TCAGTTTTAGCGGTTGGATAAATCCTCGGGAATGTGGCTCTGCTTCGGTAGA GTGTTATAGCCCGTGGGAATACAGCCAGCTGGGACTGAGGATTGCGACTTT TGTCAAGGATGCTGGCGTAATGGTTAAATGCCGCCCGTCTTGAACCCACGG ACCA.

In the present application, the ITS sequence refers that in an rDNA gene, an intergenic transcribed spacer of 5.8S rDNA and 28S rDNA is called ITS whose length and sequence change greatly, and an amplification product of this sequence is subjected to RFLP or sequence analysis, and may be used for classification and identification of different biotypes, strains, species, genera of fungi, and even differentiation of species that are very closely related.

The present application provides mutant Kluyveromyces marxianus. The biomass of the mutant Kluyveromyces marxianus is increased by more than 5%, preferably more than 10%, and further preferably 10-30%, and the protein content of the mutant Kluyveromyces marxianus is increased by more than 5%, preferably more than 10%, and further preferably 15-25%, compared with Kluyveromyces marxianus with the accession number of CCTCC No: M 2023217.

In some embodiments, the Kluyveromyces marxianus has tolerance to an organic acid, and preferably, the organic acid is lactic acid.

In the present application, the term tolerance refers to a growth condition of a strain in the presence of a substance. For example, in the case of lactic acid, tolerance refers to a growth condition of Kluyveromyces marxianus in the presence of lactic acid.

In some embodiments, a tolerance concentration of the Kluyveromyces marxianus to the lactic acid is less than 5%, preferably 1-5%.

For example, the tolerance concentration of the Kluyveromyces marxianus to the lactic acid is 5%, 4.5%, 4%, 3.5%, 2%, 2.5%, 1.5%, 1%, etc.

In the present application, both the original strain and the mutant strain have better tolerance to the lactic acid and have a tolerance concentration of 1-5.5 wt %. The mutant strain has better tolerance to the lactic acid, and may have a tolerance concentration up to 5.5 wt %. In some embodiments, the tolerance of the mutant Kluyveromyces marxianus strain is 80-90%, for example, may reach 80%, 81%, 82%, 83%, 84%, 85%, 86%, 87%, 88%, 89%, or 90%, under the condition that the lactic acid concentration is 4.5-5.5 wt %.

In some embodiments, the accession number of the mutant Kluyveromyces marxianus strain is CCTCC No: M 20222109.

In some embodiments, the Kluyveromyces marxianus has tolerance to a low pH value.

In the present application, the low pH refers to a pH value below 4.0.

In the present application, the original strain and the mutant strain have better tolerance to a low pH value, e.g., below 4.0. The mutant strain has better tolerance to low pH and can grow under the condition that pH is 2, indicating that the original strain and the mutant strain of the present application both have better tolerance to low pH.

In some embodiments, the Kluyveromyces marxianus uses an organic acid as a carbon source.

The present application provides a use of the above-mentioned Kluyveromyces marxianus with the accession number of CCTCC No: M 2023217 or the mutant Kluyveromyces marxianus in the production of microbial proteins.

The Kluyveromyces marxianus with the accession number of CCTCC No: M 2023217 or the mutant Kluyveromyces marxianus of the present application may be used in the production of microbial proteins due to high protein yield.

The present application provides a use of the above-mentioned Kluyveromyces marxianus with the accession number of CCTCC No: M 2023217 or the mutant Kluyveromyces marxianus in high-value transformation of an agricultural and forestry product. Preferably, the agricultural and forestry product is corn steep liquor.

The present application provides a microbial agent. The microbial agent contains the above-mentioned Kluyveromyces marxianus with the accession number of CCTCC No: M 2023217 or the mutant Kluyveromyces marxianus.

The present application provides a fermentation composition. The fermentation composition is obtained by fermenting the above-mentioned Kluyveromyces marxianus with the accession number of CCTCC No: M 2023217 or the mutant Kluyveromyces marxianus.

In the present application, fermentation conditions are not limited in the present application in any way. Fermentation is performed under conventional fermentation conditions in the art. For example, the fermentation can be performed at 25-37° C. for 11-60 h.

The present application provides a method for producing a microbial protein. The method includes:
fermenting the above-mentioned Kluyveromyces marxianus with the accession number of CCTCC No: M 2023217 or the mutant Kluyveromyces marxianus or the above-mentioned microbial agent.

Fermentation conditions are not limited in the present application in any way. Fermentation is performed under conventional fermentation conditions in the art. For example, the fermentation can be performed at 25-37° C. for 11-60 h.

The present application provides a food. The food contains the above-mentioned fermentation composition or the microbial protein obtained by the above-mentioned method.

The present application provides feed. The feed contains the above-mentioned fermentation composition or the microbial protein obtained by the above-mentioned method.

The present application provides a use of the above-mentioned original strain or mutant strain in feed production.

### EXAMPLES

The manufacturers of the raw materials and equipment used in the present examples, as well as the equipment and analytical methods used in the analysis of products, are described as follows. The chemical substances which are not labeled are all chemically pure grades of conventional reagents. Table 1 is a reagent information table used in the examples. Table 2 is an instrument information table used in the examples.

TABLE 1
Reagent information table

| Reagent | Manufacturer |
|---|---|
| Yeast extract | Angel Yeast Co., Ltd. |
| powder | |
| Peptone | Angel Yeast Co., Ltd. |
| Glucose | Sinopharm Chemical Reagent Co., Ltd. |
| | HuShi Reagents |
| Xylose | Sinopharm Chemical Reagent Co., Ltd. |
| | HuShi Reagents |
| Cellobiose | Sinopharm Chemical Reagent Co., Ltd. |
| | HuShi Reagents |
| Lactic acid | Sinopharm Chemical Reagent Co., Ltd. |
| | HuShi Reagents |
| Agar | Beijing Solarbio Science & Technology Co., Ltd. |

TABLE 2
Instrument information table

| Instrument | Model | Manufacturer |
|---|---|---|
| Analytical balance | ME4002E | METTLER TOLEDO |
| pH meter | PB-10 | Sartorius |
| Superclean bench | SKJH-1109 | Shanghai Sukun Industry Co., |
| | | Ltd. |
| Constant | ZWYR-2102C | Shanghai Zhicheng Analytical |
| temperature shaker | | Instrument Manufacturing Co., |
| | | Ltd. |
| Biochemical | SPX-158L | Ningbo Scientz Biotechnology |
| incubator | | Co., Ltd. |
| Centrifuge | DL-5200B- | Shanghai Anting Scientific |
| | II | Instrument Factory |
| Optical microscope | CX43 | OLYMPUS |
| Fully automatic | BioscreenC | OY Growth Curves |
| growth curve | | |
| analyzer | | |
| Electrothermal | DHG-9070A | Shanghai Jinghong Experimental |
| blowing dry box | | Equipment Co., Ltd. |

Furthermore, the mediums involved in the following examples were as follows.
YPD medium: 10 g of yeast extract powder, 20 g of glucose, 20 g of peptone, and 1000 mL of water, having pH of 4.8, all of which were sterilized at 115° C. for 20 min. Lactic acid-carbon source medium: 10 g of yeast extract powder, 20 g of peptone, and 1000 mL of water, having pH of 4.8, all of which were sterilized at 115° C. for 20 min. Lactic acid was filtered, sterilized and then added to the above-mentioned medium.
Corn steep liquor-simulated medium: 10 g of yeast extract powder, 20 g of peptone, 3 g of fructose, 64.5 g of glucose, 1.5 g of glycerin, 1 g of acetic acid, 30 g of lactic acid, and 1000 mL of water, having pH of 4.8, all of which were sterilized at 115° C. for 15 min.

### Example 1

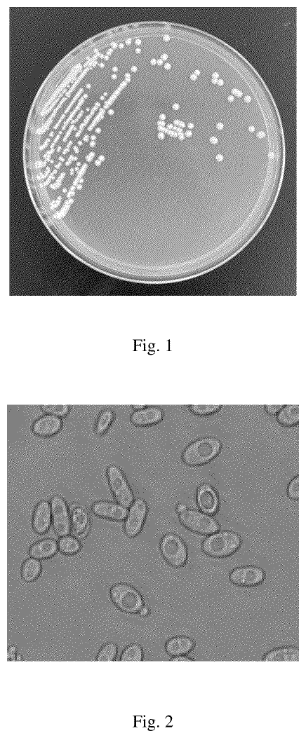

Each fermented dairy product sample collected in Xizang Autonomous Region was dissolved in sterile water and mixed well. A microbial suspension was pipetted and diluted in 10-fold series to prepare a 10⁻⁵ microbial suspension and a 10⁻⁶ microbial suspension, which were then coated in a YPD medium and cultured at 30° C. for 24-48 h. A yeast morphology was observed under a microscope, and the results were shown in FIG. 1; and meanwhile, the characteristics of single colonies on a flat plate were observed, and the results were shown in FIG. 2. It can be seen from FIG. 1 and FIG. 2, the colonies of this yeast strain had cheese-like character, milky white color, smooth surfaces, neat edges, and oval micromorphology, showing budding and reproductive properties. This yeast strain was subjected to streak purification twice, inoculated into a YPD slant medium, and stored at 4° C. A yeast strain genome was extracted.

The ITS sequences of the yeast were amplified with ITS1 (5′-TCCGTAGGTGAACCTGCGG-3′, SEQ ID NO: 1) and ITS4 (5′-TCC TCCGCTTATTGATATGC-3′, SEQ ID NO: 2) as primers. After 1% gel electrophoresis detection and sequencing, the sequences were compared with those on GenBank by Blast analysis, and the sequences with a similarity greater than 99% were considered to be from the same species. In conjunction with morphological analysis and molecular identification, this strain was Kluyveromyces marxianus with a strain number of AMCC30634. AMCC was the abbreviation of Angel Microbiological Culture Collection Center, and its ITS sequence was shown in SEQ ID NO: 3. This strain was preserved in the China Center for Type Culture Collection (CCTCC) on Feb. 27, 2023, and the accession number was CCTCC No: M 2023217.

### Example 2 Mutagenesis of Kluyveromyces marxianus

The strain AMCC 30634 obtained in Example 1 was inoculated at an inoculation amount of 2% into a test tube filled with 5 mL of YPD liquid, and cultured at 30° C., 180 rpm for 20 h. Fungi were collected by centrifugation with a centrifuge, washed once with ddH₂O, and then resuspended to a viable count of approximately 5×10⁷ CFU/mL. 10 μL of fungus was pipetted, dropped to a metal plate and then coated well. The mutagenesis conditions were as follows: a treatment distance was 2 mm, a treatment power was 120 W, an air flow rate was 10 SLM, a dispensing volume was 10 μL, and the irradiation times were 0, 5, 10, 15, 20, 25, and 30 s, respectively. Then, the fungus was put in 1 mL of normal saline, mixed well and subjected to gradient dilution. 0.1 mL of fungus was then pipetted, coated in a YPD solid medium, and cultured at 30° C. for 48 h. A lethality rate was determined according to the conventional methods in the art. From a flat plate with a mortality rate of over 98%, 98 single colonies with larger size than the original strain were picked out and inoculated into a YPD medium for activation. The 98 strains were all mutant strains, named AMCC 31342, and were graded and screened according to Example 3 to Example 8 to obtain a dominant strain. This strain was preserved in the China Center for Type Culture Collection (CCTCC) on Dec. 30, 2022, and the accession number was CCTCC No: M 20222110.

### Example 3 pH Tolerance Analysis

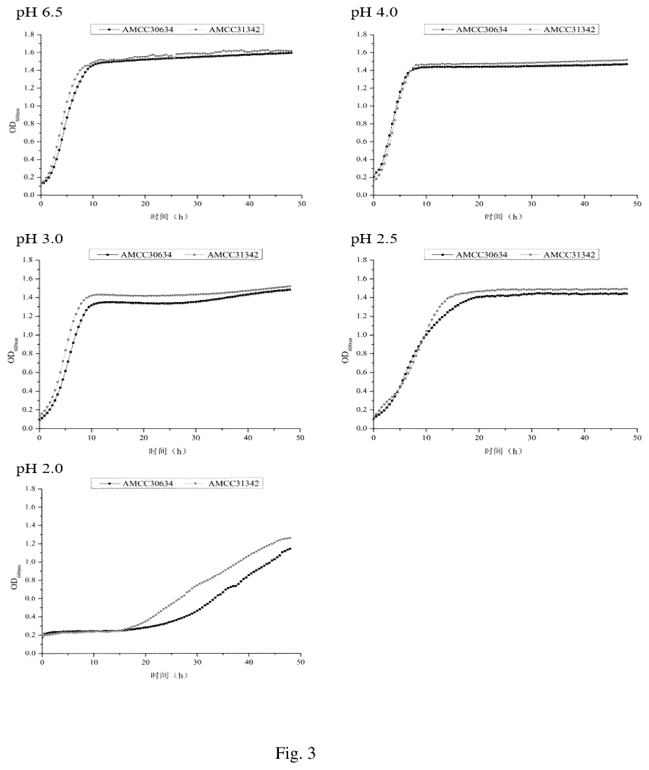

The activated microbial solution of the mutant strain obtained in Example 2 was inoculated at an inoculation amount of 3% into a 100-well culture plate filled with 300 μL of YPD mediums with different pHs (2.0, 2.5, 3.0, 4.0, 6.5), and a BioscreenC instrument was prepared for on-machine determination. The following parameters were set: the temperature was 30° C., the time was 24 h, the wavelength was 600 nm, the data was measured every 30 min, followed by determination of growth curves, and the results were shown in FIG. 3. A specific growth rate and tolerance were determined, and the results were shown in Table 3. The specific growth rate and tolerance were calculated according to the following formulas:

$$specific growth rate ( μ ) = \frac{LNOD 2 - LNOOD 2}{t 2 - t 1}$$

OD1: a corresponding OD₆₀₀ value at t1;
OD2: a corresponding OD₆₀₀ value at t2;
t2: end time of a logarithmic growth phase; and
t1: start time of the logarithmic growth phase;

$$pH tolerance ( \% ) = \frac{OD 2 - OD 1}{OD 4 - OD 3} \times 100 \%$$

OD₁—a corresponding OD₆₀₀ nm value of a strain in a YPD medium at certain pH at 0 h;
OD₂—a corresponding OD₆₀₀ nm value of a strain in a YPD medium at certain pH at 48 h;
OD₃—a corresponding OD₆₀₀ nm value of a strain in a YPD medium (natural pH of 6.5) at 0 h;
OD₄—a corresponding OD₆₀₀ nm value of a strain in a YPD medium (natural pH of 6.5) at 48 h; and

$$relative percentage ( \% ) = \frac{Content of a physiological index in screened strain}{Content of a physiological index in control strain} \times 100 \% .$$

TABLE 3
Growth indexes of strains at different pHs

| | | | Relative | | Relative | pH | Relative |
|---|---|---|---|---|---|---|---|
| | | | percentage | | percentage | tolerance | percentage |
| pH | Strain | ODₘₐₓ(nm) | (%) | μₘₐₓ(h⁻¹) | (%) | (%) | (%) |
| Natural | AMCC | 1.595 | | 0.485 | | — | |
| pH (6.5) | 30634 | | | | | | |
| | AMCC | 1.619 | 102 | 0.535 | 110 | — | |
| | 31342 | | | | | | |
| 2 | AMCC | 1.141 | | 0.08 | | 72 | |
| | 30634 | | | | | | |
| | AMCC | 1.237 | 108 | 0.116 | 145 | 76 | 106 |
| | 31342 | | | | | | |
| 2.5 | AMCC | 1.44 | | 0.371 | | 90 | |
| | 30634 | | | | | | |
| | AMCC | 1.492 | 104 | 0.439 | 118 | 92 | 102 |
| | 31342 | | | | | | |
| 3 | AMCC | 1.483 | | 0.442 | | 93 | |
| | 30634 | | | | | | |
| | AMCC | 1.521 | 103 | 0.457 | 103 | 94 | 101 |
| | 31342 | | | | | | |
| 4 | AMCC | 1.468 | | 0.462 | | 92 | |
| | 30634 | | | | | | |
| | AMCC | 1.517 | 103 | 0.487 | 105 | 94 | 102 |
| | 31342 | | | | | | |

As can be seen from FIG. 3 and Table 3, both strains could grow at pH of 2.0-6.5, and had tolerance that was higher than 90% under the condition that pH was 2.5-6.5. However, both strains grew slowly under the condition that pH was 2.0. Under this condition, the tolerance of the strain AMCC 30634 was 72%, and the tolerance of the mutant strain AMCC 31342 was 76%, which was 6% higher than that of the original strain. In practical applications, a large amount of lactic acid would be produced after fermentation of low biomass resources, resulting in decrease in pH in an environment, further indicating that the strains AMCC 30634 and AMCC 31342 could grow and produce a large number of fungi in a low pH environment. In addition, the strain AMCC 31342 had a higher potential to achieve high-value utilization of low biomass resources, and at the same time, could reduce the cost increase caused by the use of an acidic neutralizer.

### Example 4 Screening of Lactic Acid Tolerance

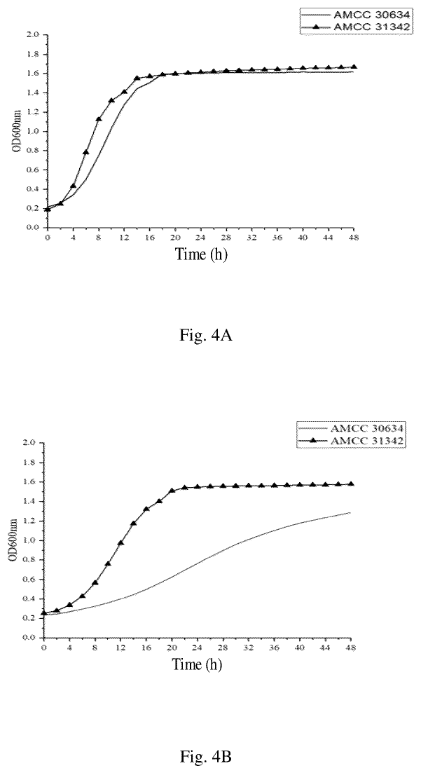

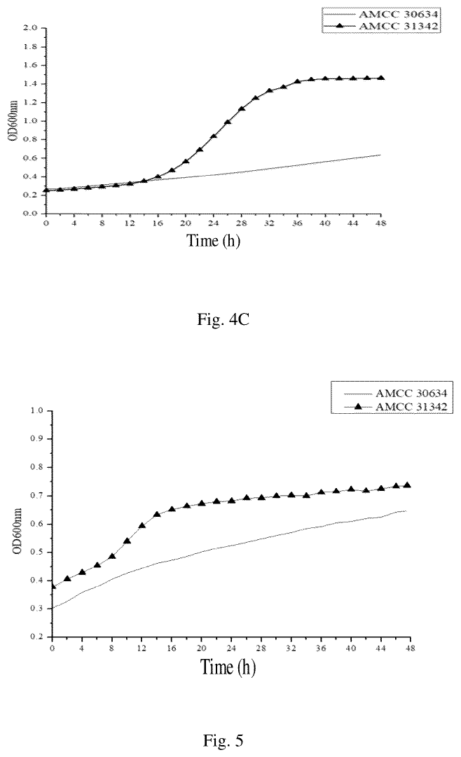

Components of corn steep liquor were detected by HPLC (after the corn steep liquor was diluted, an ion-exchange chromatography column at a column temperature of 65° C. was selected, and the components of the corn steep liquor were detected by using a differential refractive index detector as detection means). The contents of the main components were shown in Table 4. The results showed that the main carbon source in the corn steep liquor was lactic acid which had the content of about 8%. The mutant strain was screened for tolerance with 0%, 2%, 4%, 5%, and 6% lactic acids. The activated microbial solution of the mutant strain obtained in Example 2 was inoculated at an inoculation amount of 3% into a 100-well culture plate filled with 300 μLL of YPD mediums with different lactic acid concentrations, and a BioscreenC instrument was prepared for on-machine determination. The following parameters were set: the temperature was 30° C., the time was 24 h, the wavelength was 600 nm, the data was measured every 30 min, followed by determination of growth curves, and the results were shown in FIG. 4A to FIG. 4C. FIG. 4A was a schematic diagram of growth curves of the two strains in a YPD medium containing 2% lactic acid, FIG. 4B is a schematic diagram of growth curves of the two strains in a YPD medium containing 4% lactic acid, and FIG. 4C is a schematic diagram of growth curves of the two strains in a YPD medium containing 5% lactic acid. OD600 nm, a specific growth rate (μ), a lag phase, a tolerance and a relative percentage were calculated, wherein formulas for calculating the specific growth rate and the relative percentage were the same as those in Example 3, the tolerance was calculated according to the following formula, and the results were shown in Table 5 to Table 8.

$$Lactic acid tolerance ( \% ) = \frac{OD 2 - OD 1}{OD 4 - OD 3} \times 100 \%$$

TABLE 4
Content of main components in corn steep liquor (%)

| Components | Lactic acid | Total sugar | Reducing sugar |
|---|---|---|---|
| Content | 8 | 3 | 2.7 |

OD₁—a corresponding OD₆₀₀ nm value of a strain in a YPD medium at certain lactic acid concentration at 0 h;
OD₂—a corresponding OD₆₀₀ nm value of a strain in a YPD medium at certain lactic acid concentration at 48 h;
OD₃—a corresponding OD₆₀₀ nm value of a strain in a YPD medium at 0 h; and
OD₄—a corresponding OD₆₀₀ nm value of a strain in a YPD medium at 48 h.

TABLE 5
Growth indexes of strains at lactic acid concentration of 0%
(common YPD)

| | | Relative | | Relative | Lag | Relative |
|---|---|---|---|---|---|---|
| | OD600 | percentage | | percentage | phase | percentage |
| Strain | (nm) | (%) | μ(h⁻¹) | (%) | (h) | (%) |
| AMCC | 1.695 | — | 0.210 | — | 3.4 | — |
| 30634 | | | | | | |
| AMCC | 1.757 | 104 | 0.357 | 170 | 2 | 59 |
| 31342 | | | | | | |

TABLE 6
Growth indexes of strains at lactic acid concentration of 2% (pH of 3.06)

| | | | | | | | Lactic | |
|---|---|---|---|---|---|---|---|---|
| | | Relative | | Relative | | Relative | acid | Relative |
| | OD600 | percentage | μ | percentage | Lag phase | percentage | tolerance | percentage |
| Strain | (nm) | (%) | (h⁻¹) | (%) | (h) | (%) | (%) | (%) |
| AMCC | 1.616 | — | 0.210 | — | 4 | — | 95 | — |
| 30634 | | | | | | | | |
| AMCC | 1.877 | 116 | 0.316 | 150 | 2 | 50 | 107 | 113 |
| 31342 | | | | | | | | |

TABLE 7
Growth indexes of strains at lactic acid concentration of 4% (pH of 2.72)

| | | | | | | | Lactic | |
|---|---|---|---|---|---|---|---|---|
| | | Relative | | Relative | | Relative | acid | Relative |
| | OD600 | percentage | μ | percentage | Lag | percentage | tolerance | percentage |
| Strain | (nm) | (%) | (h⁻¹) | (%) | phase (h) | (%) | (%) | (%) |
| AMCC | 1.289 | — | 0.064 | — | 7.4 | — | 76 | — |
| 30634 | | | | | | | | |
| AMCC | 1.577 | 122 | 0.150 | 234 | 4 | 54 | 90 | 118 |
| 31342 | | | | | | | | |

TABLE 8
Growth indexes of strains at lactic acid concentration of 5% (pH of 2.63)

| | | Relative | | Relative | | Relative | Lactic acid | Relative |
|---|---|---|---|---|---|---|---|---|
| | OD600 | percentage | μ | percentage | Lag phase | percentage | tolerance | percentage |
| Strain | (nm) | (%) | (h⁻¹) | (%) | (h) | (%) | (%) | (%) |
| AMCC | 0.636 | — | 0.034 | — | 16 | — | 38 | — |
| 30634 | | | | | | | | |
| AMCC | 1.464 | 230 | 0.103 | 303 | 12 | 75 | 83 | 218 |
| 31342 | | | | | | | | |

As can be seen from the results of FIG. 4A to FIG. 4C and Table 5 to Table 8, both AMCC 30634 and AMCC 31342 had tolerance to lactic acid and had a phenomenon of secondary growth. The maximum tolerance concentration of lactic acid was 5% lactic acid (an OD600 increase of more than 50% was considered to be growth); and if the lactic acid concentration was greater than 5%, the growth of yeast strains was inhibited. When the lactic acid concentration was 0-5%, the biomass (OD600) and specific growth rate of the mutant strain AMCC 31342 were both higher than those of the original strain AMCC 30634, and the lag phases of the mutant strain AMCC 31342 and the original strain AMCC 30634 were both advanced. When the lactic acid concentration was 2%, the tolerance of the AMCC 31342 strain was greater than 100%, indicating that the lactic acid concentration of 2% could promote its growth without any inhibiting effect, which was the optimal lactic acid growth concentration of the strain and could be applied to the actual fermentation production. When the lactic acid concentration was 5%, the biomass of the AMCC 31342 strain was increased by 130%, the specific growth rate was increased by 203%, the lag phase was shortened by 25% and the tolerance was increased by 118% compared with AMCC 30634.

### Example 5 Analysis of Lactic Acid Utilization and Growth

The mutant strain and the original strain obtained in Example 2 were respectively inoculated into a 100-well culture plate filled with 300 μL of 2% lactic acid-carbon source medium, and a BioscreenC instrument was prepared for on-machine determination. The following parameters were set: the temperature was 30° C., the time was 24 h, the wavelength was 600 nm, the data was measured every 30 min, followed by determination of growth curves, and the results were shown in FIG. 5. The OD600 nm was calculated, the specific growth rate (μ) and relative percentage were calculated according to the formula in Example 3, and the lag phase were calculated. The results were shown in Table 9.

TABLE 9
Growth indexes of strains under 2%
lactic acid-carbon source medium

| | | Relative | | Relative | Lag | Relative |
|---|---|---|---|---|---|---|
| | OD600 | percentage | | percentage | phase | percentage |
| Strain | (nm) | (%) | μ(h⁻¹) | (%) | (h) | (%) |
| AMCC | 0.647 | — | 0.067 | — | 8 | — |
| 30634 | | | | | | |
| AMCC | 0.737 | 114 | 0.069 | 103 | 6 | 75 |
| 31342 | | | | | | |

As can be seen from FIG. 5 and Table 9, both AMCC 30634 and AMCC 31342 strains could use lactic acid as a carbon source for growth, and the biomass of the mutant strain AMCC 31342 was increased by 14%, the specific growth rate was increased by 3% and the lag phase was shortened by 25% compared with AMCC 30634, further indicating that after mutagenesis, the lactic acid utilization capacity of AMCC 31342 was improved, and AMCC 31342 can be transformed into a yeast strain that can utilize lactic acid efficiently.

### Example 6 Small-System Screening of Corn Steep Liquor-Simulated Medium (Screening in 100-Well Culture Plate)

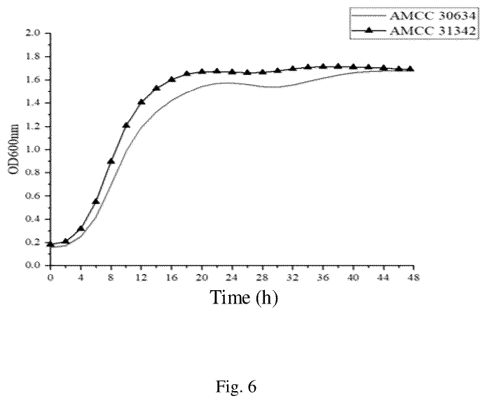

The mutant strain and the original strain obtained in Example 2 were respectively inoculated at an inoculation amount of 3% into a 100-well culture plate filled with 300 μL of corn steep liquor-simulated medium, and a BioscreenC instrument was prepared for on-machine determination. The following parameters were set: the temperature was 30° C., the time was 24 h, the wavelength was 600 nm, the data was measured every 30 min, followed by determination of growth curves, and the results were shown in FIG. 6. The OD600 nm was calculated, the specific growth rate (p) and relative percentage were calculated according to the formula in Example 3, and the lag phase were calculated. The results were shown in Table 10.

TABLE 10
Growth indexes of strains under corn
steep liquor-simulated medium

| | | Relative | | Relative | Lag | Relative |
|---|---|---|---|---|---|---|
| | OD600 | percentage | | percentage | phase | percentage |
| Strain | (nm) | (%) | μ(h⁻¹) | (%) | (h) | (%) |
| AMCC | 1.679 | — | 0.276 | — | 4 | — |
| 30634 | | | | | | |
| AMCC | 1.692 | 101 | 0.290 | 105 | 3.5 | 88 |
| 31342 | | | | | | |

As can be seen from FIG. 6 and Table 10, the AMCC 30634 and AMCC 31342 strains could grow normally in a corn steep liquor system, and the specific growth rate of the mutant strain AMCC 31342 was increased by 5% and the lag phase was shortened by 12% compared with AMCC 30634, indicating that AMCC 31342 entered the logarithmic growth phase faster and had more efficient utilization efficiency of corn steep liquor.

### Example 7 Shake Flask Fermentation Test of Corn Steep Liquor-Simulated Medium

The mutant strain and the original strain obtained in Example 2 were inoculated at an inoculation amount of 0.5% into a shake flask filled with 300 mL of corn steep liquor-simulated medium, shaken at 30° C., cultured overnight, and centrifugally washed to obtain yeast milk. A rapid moisture analyzer was used to perform moisture detection of the yeast milk, a dry weight (the biomass was a weight of the yeast milk after centrifugation, and could be weighed directly: dry weight (g/L)=biomass (g/L)*(1−moisture %) of the yeast milk was calculated, and the relative percentage was calculated according to the calculation formula in Example 3. The results were shown in Table 11. A method for determining a protein was as follows:

1 g of yeast milk (accurate to 0.0002 g) was weighed accurately and placed into a digestive tube, added with 2.5 g of digestive powder, slowly added with 10 mL of concentrated sulfuric acid along a wall of the tube, then placed on a digestion device, and digested for about 3 h until it is smokeless; the solution became clear and light yellow, and then continued to be heated for 10 min. The digestive tube was taken out, placed for cooling, rinsed for the tube wall with about 30 mL of distilled water, cooled again, then transferred into a 100 mL volumetric flask, rinsed with a small amount of water for three times, poured into a volumetric flask, added with water to a constant volume scale, and shaken well to obtain a digestive solution for later use. 25 mL of the digestive solution was pipetted accurately into the digestive tube and placed on a distillation device. 25 mL of boric acid solution was added to a triangular flask, added with 4-6 drops of methyl red-bromocresol green mixed indicator as a receiving solution and placed on a receiving table. A circulating water valve was opened; 25 mL of sodium hydroxide solution was added to the digestive tube; a steam switch was switched on, and the receiving table was lift, such that a receiving tube was immersed in the receiving solution and distilled till the receiving solution was 150-200 mL; the receiving table was put down, and steam and circulating water was turned off; a receiving nozzle was rinsed with distilled water, and the receiving bottle was taken out. The receiving solution was titrated with a 0.05 mol/L sulfuric acid standard solution; an end point was reached as the color became reddish from green; a blank test was done at the same time according to the above method; and the protein content was calculated according to the following formula:

X=(C(V₁−V₂)×0.01401)/(W×Ds×25/100)×100×6.25
in which: X represented a percentage of the protein in the sample, %;
C represented a concentration of the sulfuric acid standard solution, mol/L;
V₁ represented a volume of the sulfuric acid standard solution consumed by the titration of the sample, mL;
V₂ represented a volume of the sulfuric acid standard solution consumed by the titration of a control, mL;
0.01401 referred to a mass of nitrogen expressed in grams equivalent to 1.00 mL of sulfuric acid [C(½H2SO4)=1.000 mol/L] standard solution;
W represented a mass of the sample, g;
Ds represented a percentage of a dry substance in the sample, %; and
6.25 represented a conversion factor of nitrogen to crude protein.

TABLE 11
Fermentation indexes for different strains

| | | Relative | Dry | Relative | Pro- | Relative |
|---|---|---|---|---|---|---|
| | Biomass | percentage | weight | percentage | tein | percentage |
| Strain | (g/L) | (%) | (g/L) | (%) | (%) | (%) |
| AMCC | 10.48 | — | 2.96 | — | 51.0 | — |
| 30634 | | | | | | |
| AMCC | 13.33 | 127 | 3.94 | 133 | 51.3 | 101 |
| 31342 | | | | | | |

The results showed that the biomass, dry weight and protein content indexes of the mutant strain AMCC 30634 were higher than those of the original strain AMCC 30634. The biomass was increased by 27% compared with the original strain AMCC 30634, the dry weight was increased by 33% compared with the original strain AMCC 30634, and the protein content was increased by 1% compared with the original strain AMCC 30634, further indicating that AMCC 30634 could be produced normally in an acidic environment of corn steep liquor and could be efficiently transformed into microbial proteins using the corn steep liquor.

### Example 8 Small-Scale Fermentation Test of Corn Steep Liquor

A small-scale fermentation test was carried out on AMCC 30634 and AMCC 31341.

(1) whole corn steep liquor fermentation: a domestic Baoxing 45 L fermenter was selected for fed-batch fermentation, and a fed-batch medium contained 100% of corn steep liquor, and was sterilized at 115° C. for 20 min. Fermentation was performed for 14 h. The pH was controlled at 4.3-5.6 and the culture temperature was 30-32° C. in the fermentation process. At the end of fermentation, fermentation broth was centrifuged, followed by suction filtration. The weight of the yeast milk was weighed and the moisture of the yeast milk was detected (with a rapid moisture detector). The dry weight of the yeast milk was calculated. The volume of the fermentation broth and the weight of corn steep liquor were measured. The fermentation indexes of the two strains were compared according to the following formula, and the results were shown in Table 12, wherein the unit consumption was defined as: grams of corn steep liquor required to produce 1 g of dry yeast/protein.

Unit consumption of yeast (g)=weight of corn steep liquor/dry weight of produced yeast

Unit consumption of protein (g)=unit consumption of yeast*protein content (a protein measurement method was the same as that in Example 7)

yeast concentration (g/L)=weight of yeast milk/empty fermenter volume

Microbial protein concentration (g/L)=yeast concentration*protein content

Microbial protein (g)=dry weight*protein content

TABLE 12
Small-scale fermentation indexes of whole corn steep liquor

| | Unit | Unit | Empty | | | | Microbial | |
|---|---|---|---|---|---|---|---|---|
| | consumption | consumption | fermenter | yeast | Dry | Protein | protein | Microbial |
| | of yeast | of protein | volume | concentration | weight | content | concentration | protein |
| Strain | (g) | (g) | (L) | (g/L) | (g) | (%) | (g/L) | (g) |
| AMCC | 38.37 | 67.08 | 21.8 | 125 | 558 | 53.8 | 67 | 300 |
| 30634 | | | | | | | | |
| AMCC | 33.7 | 62.69 | 28 | 110 | 631 | 57.2 | 63 | 361 |
| 31342 | | | | | | | | |

(2) Sugar-supplemented fermentation of corn steep liquor a domestic Baoxing 45 L fermenter was selected for fed-batch fermentation, and a fed-batch medium contained 50% of corn syrup and 50% of glucose (in percentage by weight), and was sterilized at 115° C. for 20 min. Fermentation was performed for 14 h. The pH was controlled at 4.3-5.6 and the culture temperature was 30-32° C. in the fermentation process. The fermentation indexes of the two strains were compared, and the results were shown in Table 13. (Unit consumption: grams of carbon source required to produce 1 g of dry yeast/protein.)

TABLE 13
Small-scale sugar-supplemented fermentation indexes of corn steep liquor

| | Unit | Unit | Empty | | | | Microbial | Microbial |
|---|---|---|---|---|---|---|---|---|
| | consumption | consumption | fermenter | yeast | Dry | Protein | protein | protein |
| | of yeast | of protein | volume | concentration | weight | content | concentration | (g) |
| Strain | (g) | (g) | (L) | (g/L) | (g) | (%) | (g/L) | dry |
| AMCC | 4.7 | 8.0 | 17.6 | 310 | 1118 | 58.9 | 183 | 659 |
| 30634 | | | | | | | | |
| AMCC | 4.4 | 7.3 | 19 | 300 | 1169 | 60 | 180 | 701 |
| 31342 | | | | | | | | |

In a fermentation system of whole corn steep liquor, lactic acid was used as a main carbon source, and the two strains could grow normally, indicating that the AMCC 31342 and AMCC 31342 strains had tolerance to the lactic acid and could be transformed into microbial proteins by using lactic acid. The unit consumption of yeast and the unit consumption of protein were compared. The AMCC 31342 strain had more efficient lactic acid utilization capacity, the dry weight of the mutant strain AMCC 31342 was increased by 13% compared with the original strain, and the microbial protein of the mutant strain AMCC 31342 was increased by 20% compared with the original strain.

In a sugar-supplemented fermentation system of corn steep liquor, glucose and lactic acid were used as a carbon source, AMCC 31342 had lower unit consumption, the dry weight index of AMCC 31342 was increased by 5% compared with the original strain, and the microbial protein of AMCC 31342 was increased by 7% compared with the original strain. The dry weight and protein indexes of the two strains were higher than those in whole corn steep liquor fermentation, indicating that the production efficiency was higher after sugar supplementation, that is, there was still some room for optimization in the fermentation process.

It was further explained that the two strains in the present invention could achieve high-value utilization of low-cost biomass, and were used in the field of foods or feed proteins as alternative proteins to reduce the pressure on environmental resources brought by the traditional aquaculture industry, alleviate the protein requirements of global population and the field of feed, and achieve the economic development and environmental protection of the fermentation industry.

The foregoing descriptions are merely preferred examples of the present invention, and are not intended to limit the present invention in any form. Any modifications, equivalent substitutions, improvements, etc. made within the spirit and principles of the present invention should fall within the protection scope of the present invention.

Sequence listings

| SEQ ID | Sequence | |
|---|---|---|
| NOs: | description | Sequences |
| SEQ ID | Primer | TCCGTAGGTGAACCTGCGG |
| NO: 1 | sequence | |
| SEQ ID | Primer | TCC TCCGCTTATTGATATGC |
| NO: 2 | sequence | |
| SEQ ID | ITS | CCAACGGGGATTGCCTTAGTACGGCGAGTGAAGCGGCAAAA |
| NO: 3 | sequence of | GCTCAAATTTGAAATCTGGCGTCTTCGACGTCCGAGTTGTAA |
| | original | TTTGAAGAAGGCGACTTTGTAGCTGGTCCTTGTCTATGTTCCT |
| | strain | TGGAACAGGACGTCATAGAGGGTGAGAATCCCGTGTGGCGA |
| | | GGATCCCAGTTATTTGTAAAGTGCTTTCGACGAGTCGAGTTG |
| | | TTTGGGAATGCAGCTCTAAGTGGGTGGTAAATTCCATCTAAA |
| | | GCTAAATATTGGCGAGAGACCGATAGCGAACAAGTACAGTGA |
| | | TGGAAAGATGAAAAGAACTTTGAAAAGAGAGTGAAAAAGT |
| | | ACGTGAAATTGTTGAAAGGGAAGGGCATTTGATCAGACATG |
| | | GCGTTTGCTTCGGCTTTCGCTGGGCCAGCATCAGTTTTAGCG |
| | | GTTGGATAAATCCTCGGGAATGTGGCTCTGCTTCGGTAGAGT |
| | | GTTATAGCCCGTGGGAATACAGCCAGCTGGGACTGAGGATTG |
| | | CGACTTTTGTCAAGGATGCTGGCGTAATGGTTAAATGCCGCC |
| | | CGTCTTGAACCCACGGACCA |

1. A Kluyveromyces marxianus strain having an accession number of China Center for Type Culture Collection (CCTCC) No: M 2023217.

2. A mutant Kluyveromyces marxianus strain derived from the Kluyveromyces marxianus according to claim 1, characterized in that the biomass of the mutant Kluyveromyces marxianus is increased by more than 5%, compared with the Kluyveromyces marxianus according to claim 1.

3. The mutant Kluyveromyces marxianus according to claim 2, characterized in that the protein content of the mutant Kluyveromyces marxianus is increased by more than 5%, compared with the Kluyveromyces marxianus according to claim 1.

4. The mutant Kluyveromyces marxianus according to claim 2, characterized in that the accession number of the mutant Kluyveromyces marxianus is CCTCC No: M 20222110.

5. The mutant Kluyveromyces marxianus according to claim 2, characterized in that the mutant Kluyveromyces marxianus has tolerance to an organic acid.

6. The mutant Kluyveromyces marxianus according to claim 2, characterized in that a tolerance concentration of the mutant Kluyveromyces marxianus to the lactic acid.

7. The mutant Kluyveromyces marxianus according to claim 2, characterized in that the mutant Kluyveromyces marxianus has tolerance to a low pH value.

8. The mutant Kluyveromyces marxianus according to claim 2, characterized in that the mutant Kluyveromyces marxianus uses an organic acid as a carbon source.

9.-11. (canceled)

12. A fermentation composition, which is obtained by fermenting the Kluyveromyces marxianus according to claim 1.

13.-16. (canceled)

17. The Kluyveromyces marxianus according to claim 1, characterized in that the Kluyveromyces marxianus has tolerance to an organic acid.

18. The Kluyveromyces marxianus according to claim 1, characterized in that the Kluyveromyces marxianus has tolerance to lactic acid.

19. The Kluyveromyces marxianus according to claim 1, characterized in that a tolerance concentration of the Kluyveromyces marxianus to lactic acid is less than 5.5 wt %.

20. The Kluyveromyces marxianus according to claim 1, characterized in that a tolerance concentration of the Kluyveromyces marxianus to lactic acid is 1-5.5 wt %.

21. The Kluyveromyces marxianus according to claim 1, characterized in that the Kluyveromyces marxianus has tolerance to a low pH value.

22. The Kluyveromyces marxianus according to claim 1, characterized in that the Kluyveromyces marxianus uses an organic acid as a carbon source.

23. The mutant Kluyveromyces marxianus according to claim 2, characterized in that the biomass of the mutant Kluyveromyces marxianus is increased by 10-30%, compared with the Kluyveromyces marxianus according to claim 1.

24. The mutant Kluyveromyces marxianus according to claim 2, characterized in that the protein content of the mutant Kluyveromyces marxianus is increased by 15-25%, compared with the Kluyveromyces marxianus according to claim 1.

25. The mutant Kluyveromyces marxianus according to claim 2, characterized in that the mutant Kluyveromyces marxianus has tolerance to lactic acid.

26. The mutant Kluyveromyces marxianus according to claim 2, characterized in that a tolerance concentration of the mutant Kluyveromyces marxianus to the lactic acid is 1-5.5 wt %.

27. The mutant Kluyveromyces marxianus according to claim 4, characterized in that the protein content of the mutant Kluyveromyces marxianus is increased by more than 5%.

28. The Kluyveromyces marxianus according to claim 1 is used for preparing into food or feed comprising bacteria protein, the bacteria protein being obtained through the fermentation of the Kluyveromyces marxianus.
